SEQUENCE LISTING (2 sequences)

SEQ ID NO: 1 - DNA, 840 bp, Artificial Sequence
  RNAIII activating protein (RAP)
atggctattaaaaagtataagccaataacaaatggtcgtcgtaatatgacttcgttagatttcgcagaaatcacgaaaactacacctgaaaagtcattattaaaaccgctaccgaaaaaagcgggacgtaacaaccaaggtaaattgactgtaagacaccatggtggtggacacaaacgtcaataccgtgttatcgatttcaaacgtaacaaagatggtatcaatgcaaaagttgattctattcaatatgatccaaaccgctcagcaaacatcgctttagttgtatatgcagacggtgaaaaacgaatatatcattgcattgctcctaaaggattagaagtaggtcaaatcgttgaaagtggtgctgaagctgacactaaagttggtaacgcattaccattacaaaacattccagttggtacagtagtacacaacatcgagcttaaacctggtaaaggtggacaaatcgctcgttcagctggtgcaagtgctcaagtacttggtaaagaaggtaaatacgtattaatcagattaagatctggtgaagttcgtatgatcttatctacttgccgtgctacaatcggtcaagttggtaacctacaacacgaattagttaacgttggtaaagccggacgttcaagatggaaaggtatccgtccaacagttcgtggttctgtaatgaaccctaacgatcacccacacggtggtggtgaaggtcgtgctcctatcggtagaccatctccaatgtcaccatggggtaaacctacgcttggtaagaaaactcgtcgtggtaaaaaatcatcagacaaacttatcgttcgtggacgtaagaaaaaataa

SEQ ID NO: 2 - protein, 279 aa, Artificial Sequence
  RNAIII activating protein (RAP)
MAIKKYKPITNGRRNMTSLDFAEITKTTPEKSLLKPLPKKAGRNNQGKLTVRHHGGGHKRQYRVIDFKRNKDGINAKVDSIQYDPNRSANIALVVYADGEKRIYHCIAPKGLEVGQIVESGAEADTKVGNALPLQNIPVGTVVHNIELKPGKGGQIARSAGASAQVLGKEGKYVLIRLRSGEVRMILSTCRATIGQVGNLQHELVNVGKAGRSRWKGIRPTVRGSVMNPNDHPHGGGEGRAPIGRPSPMSPWGKPTLGKKTRRGKKSSDKLIVRGRKKK

(19) United States
(12) Patent Application Publication
(10) Pub. No.: US 2008/0219976 A1
(43) Pub. Date: Sep. 11, 2008

(54) METHODS AND COMPOSITIONS FOR TREATMENT AND PREVENTION OF STAPHYLOCOCCAL INFECTIONS

(76) Inventor: NAOMI BALABAN, Hopkinton, MA (US)

Correspondence Address: JAGTIANI + GUTTAG, 10363-A DEMOCRACY LANE, FAIRFAX, VA, 22030, US

(21) Appl. No.: 11/923,865

(22) Filed: Oct. 25, 2007

Related U.S. Application Data
(63) Continuation-in-part of application No. 11/414,350, filed on May 1, 2006.
Application No. 11/414,350 is a continuation-in-part of application No. 10/358,448, filed on Feb. 3, 2003, now Pat. No. 7,323,179.
Application No. 10/358,448 is a continuation-in-part of application No. 09/839,695, filed on Apr. 19, 2001, now Pat. No. 7,067,135.
Application No. 09/839,695 is a continuation-in-part of application No. 09/054,331, filed on Apr. 2, 1998, now Pat. No. 6,291,431.
(60) Provisional application No. 60/068,094, filed on Dec. 19, 1997.

(51) Int. Cl. A61K 39/395; A61K 38/16; C07K 16/00; A61P 31/04

(52) U.S. Cl. 424/133.1; 514/12; 424/141.1; 530/387.1; 530/388.1; 530/387.3

(57) ABSTRACT

The invention features methods and compositions for treatment or prevention of infection by, or disease caused by infection with, Staphylococcus spp., particularly S. aureus. The treatment is effected by providing to the infection target a n amount of antibody, or antibody binding fragment, which binds specifically to RNAIII activating protein (RAP) which upregulates expression of virulence factors by a Staphylococcus infection in said host, or by raising a protective immune response in said host in the form of a circulating antibody titre through administration of RAP, or a fragment thereof, which promotes the expression of antibodies thereto by said host, which antibodies bind RAP and inhibit its ability to upregulate the virulence factors.

### CROSS REFERENCE TO RELATED CASES

This application is a continuation-in-part of U.S. application Ser. No. 11/414,350, filed May 1, 2006, which is a continuation-in-part of U.S. application Ser. No. 10/358,448, filed Feb. 3, 2003, which is a continuation-in-part of U.S. application Ser. No. 09/839,695, filed Apr. 19, 2001, now U.S. Pat. No. 7,067,135, each of which is incorporated by reference in its entirety herein for all purposes.

### BACKGROUND

1. Technical Field

The present invention relates to methods and compositions for treatment or prevention of bacterial infection, diseases or symptoms caused by bacterial infection, and particularly those associated with infection by Staphylococcus spp.

2. Background of the Technology

Staphylococcus aureus causes disease chiefly through the production of virulence factors such as hemolysins, enterotoxins, and toxic shock syndrome toxin. The synthesis of virulence factors in S. aureus is controlled by a regulatory mRNA molecule, RNAIII (Novick et al., EMBO J. 12: 3967-75 (1993), Balaban et al., FEMS Microbiol. Letts. 133: 155-61 (1995), Moerfeldt et al., EMBO J. 14: 4569-77 (1995)), encoded by the agr locus that also contains agrA, agrB, agrC, and agrD. The agrA and agrC genes encode for a classical two-component signal transduction pathway, with agrC encoding a signal receptor and agrA the response regulator. agrD encodes for an octapeptide pheromone that autoinduces the agr system by phosphorylating AgrC. RNAIII is produced in culture only from the mid-exponential phase of growth and is autoinduced by the protein “RNAIII activating protein” (RAP) (Balaban et al., Proc. Nat'l Acad. Sci. USA 92: 1619-23 (1995)). RAP is continuously secreted by the bacteria and activates the agr at a concentration threshold (id.).

The autoinducers of RNAIII that have been described to date include the agr-independent RAP (Balaban et al., (1995), supra), and the agrD-derived octapeptide pheromone (Ji et al., Proc. Nat'l Acad. Sci. USA 92: 12055-59 (1995)). The agrD-derived octapeptide also has been shown to be part of a “bacterial interference” system that provides a mechanism for different S. aureus strains to compete with each other at an infection site (Ji et al., Science 276: 2027-30 (1997)). In this bacterial interference system, the octapeptide activates RNAIII transcription of the strain by which it is produced, while also acting as an inhibitor of RNAIII transcription of other strains of Staphylococcus.

In vivo, it is assumed that S. aureus first produce proteins that facilitate bacterial binding to host cells as well as the secreted autoinducer molecules. As the bacterial colony increases in density, the autoinducer molecules accumulate. Upon reaching a threshold concentration, the autoinducers activate RNAIII transcription, which in turn results in virulence factor production. The virulence factors damage and eventually destroy surrounding host cells, which serve as nutritive sources for the S. aureus bacteria and promote further growth of the colony. Thus, inhibition of RNAIII by suppression of the autoinducers or their receptors is of particular interest in treatment or prevention of S. aureus-mediated disease. Several mechanisms for RNAIII inhibition have been identified, including inhibition of RNAIII and toxin production by anti-RAP antibodies (Balaban et al., Autoinducer of virulence as a target for vaccine and therapy against Staphylococcus aureus, Science. 1998 Apr. 17; 280(5362):438-40), a RAP binding protein (Yang et al., Inhibition of Staphylococcus aureus pathogenesis in vitro and in vivo by RAP-binding peptides, Peptides. 2003 November; 24(11):1823-8; Yang et al., A novel peptide isolated from phage library to substitute a complex system for a vaccine against staphylococci infection, Vaccine. 2006 Feb. 20; 24(8):1117-23. Epub 2005 Sep. 23. PMID: 16359760 [PubMed—indexed for MEDLINE]), and by a peptide termed the “RNAIII inhibiting peptide” (RIP), which competes with RAP (Balaban et al. (1995), supra; Gov et al., RNAIII inhibiting peptide (RIP), a global inhibitor of Staphylococcus aureus pathogenesis: structure and function analysis, Peptides. 2001 October; 22(10): 1609-20; Balaban et al., Regulation of Staphylococcus aureus pathogenesis via Target of RNAIII-Activating Protein (TRAP), J. Biol. Chem. 276, 2658-2667 (2001); and Balaban et al., Prevention of staphylococcal biofilm-associated infections by the quorum sensing inhibitor RIP, Clin Orthop Relat Res. 2005 August; (437):48-54. S. aureus causes diseases ranging from minor skin infections to life-threatening deep infections such as pneumonia, endocarditis, meningitis, post-operative wound infections, septicemia, and toxic shock syndrome (Silverstein et al., in “Microbiology,” Davis et al., eds., Lippincott, Philadelphia, pp. 485-506 (1990)). Hospitalized patients are at particular risk, with over 500,000 nosocomial infections per year (Lowy, Staphylococcus aureus infections, N Engl J Med. 1998 Aug. 20; 339(8):520-32. Review. No abstract available) (Panlilio, et al., Infect. Control Hosp. Epidemiol. 13: 582-86 (1992); Lowy, Antimicrobial resistance: the example of Staphylococcus aureus, J Clin Invest. 2003 May; 111(9):1265-73). The emergence of drug resistance has made many of the available antimicrobial agents ineffective. Therefore, alternative methods for the prevention and treatment of bacterial infections in general and S. aureus infections in particular are eagerly sought. The instant invention addresses this need and others.

### SUMMARY

The invention features methods and compositions for treatment or prevention of infection by, or disease caused by infection with, Staphylococcus spp., particularly S. aureus. One aspect of the invention is a composition and a method for preventing a staphylococcal infection in a host, wherein the composition is administered to the host. In some embodiments the host is a human patient. In further embodiments the host is an animal, such as but not limited to an experimental animal.

A further aspect of the invention is a composition and a method for preventing a staphylococcal infection in a host, wherein an antagonist of or a molecule capable of binding RAP is administered to the host. In some embodiments the host is a human patient. In further embodiments the host is an animal, such as but not limited to an experimental animal. In some embodiments the antagonist is a polypeptide, a functional fragment, an adjuvant and a functional fragment, a peptidomimetic, or an antibody.

A further aspect of the invention is a composition and a method for preventing a staphylococcal infection in a host, wherein a molecule capable of binding RAP is administered to the host. In some embodiments the host is a human patient. In further embodiments the host is an animal, such as but not limited to an experimental animal. In some embodiments the binding molecule is a polypeptide, a functional fragment, an adjuvant and a functional fragment or an antibody.

### BRIEF DESCRIPTION OF THE DRAWINGS

FIG. 1 is a photograph of gel-filtration-purified RAP from wild-type (lane 1) and from agr-null strain (lane 2) separated on SDS PAGE and silver stained. Approximate molecular weight markers are indicated.

FIG. 2A is a photograph of an immunoblot of sera of vaccinated and control animals. Post-exponential supernatant of wild-type S. aureus (lanes 1/2) or purified RAP (lane 3) was separated on SDS 12% PAGE, Western blotted, and membranes were incubated in the presence of: Lane 1: pre-immune (lane 1a) or post-immune (lane 1b) sera collected from a control CFA-injected animal (diluted 1:20). Lane 2: pre-immune sera (lane 2a diluted 1:20) or post immune sera (lane 2b diluted 1:1000 and lane 3 diluted 1:20). Approximate molecular weight markers are indicated in kilodaltons.

FIG. 2B is a graph depicting the titer of anti-RAP antibodies versus lesion size of vaccinated animals.

FIG. 3 is a graph depicting the inhibition of RNAIII by purified and synthetic RIP.

FIG. 4 is a schematic illustrating the DNA (SEQ ID NO: 12) and amino acid (SEQ ID NO: 13) sequences of RAP.

FIGS. 5A and 5B are photographs of gels showing purification of recombinant RAP (rRAP) eluted from a nickel column by 1M (lane 1), 2M (lane 2) and 3M (lane 3) imidazole applied to SDS 12.5% PAGE. The gel was Western blotted, membrane stained in ponceau to visualize proteins (FIG. 6A), blocked in milk, and incubated with anti-histidine antibodies (FIG. 6B). Bound antibodies were detected by peroxidase-conjugated anti-mouse antibodies and visualized by ECL (Amersham). Molecular mass is indicated in kD.

FIG. 6 is a graph showing the percent mortality of Balb/C naive mice (control) or mice vaccinated with rL2, and challenged with 2×10⁹ S. aureus.

FIG. 7 is a graph showing the development of lesions in rL2-vaccinated animals that survived a challenge of 2×10⁹ S. aureus.

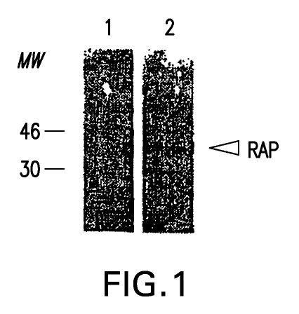

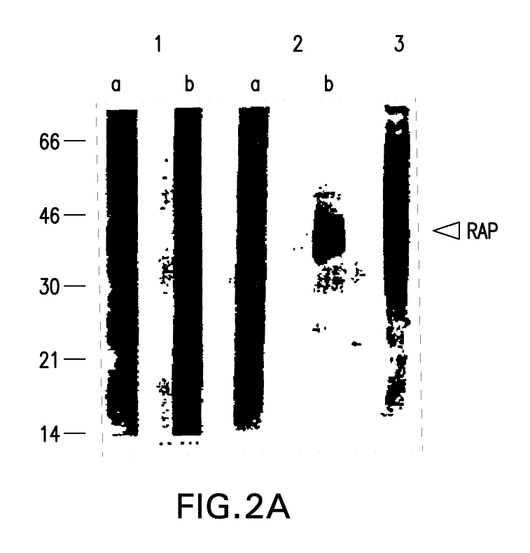

### DETAILED DESCRIPTION

Before the present proteins, formulations and methods are described, it is to be understood that this invention is not limited to the particular compounds, characteristics and steps described, as such may, of course, vary. It is also to be understood that the terminology used herein is for the purpose of describing particular embodiments only, and is not intended to limit the scope of the present invention which will be limited only by the appended claims.

All publications and patents mentioned herein are incorporated herein by reference to disclose and describe the specific methods and/or materials in connection with which the publications and patents are cited. The publications and patents discussed herein are provided solely for their disclosure prior to the filing date of the present application. Nothing herein is to be construed as an admission that the present invention is not entitled to antedate such publication or patent by virtue of prior invention. Further, the dates of publication or issuance provided may be different from the actual dates which may need to be independently confirmed.

Generally, the nomenclature used hereafter, and the laboratory procedures in cell culture and protein biochemistry are those well known and commonly employed in the art. Generally, enzymatic reactions and column chromatography are performed according the manufacturer's specifications. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For the purposes of the present invention, the foregoing terms are defined below.

The terms “pharmaceutically acceptable” or “therapeutically acceptable” refer to a substance which does not interfere with the effectiveness or the biological activity of the active ingredients and which is not toxic to the host or the patient.

The term “adjuvant” or “vaccine adjuvant” refers generally to a substance added to a vaccine (i.e. combined with an antigenic molecule) to improve a host's immune response so that a less immunogenic antigen or antigenic fragment still elicits a robust immunological response from the host (i.e. host production of a circulating titre of antibodies sufficient to inhibit biological activity on the part of the antigen).

The terms “encoding” or “encodes” refer generally to the sequence information being present in a translatable form, usually operably linked to a promoter. A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code. See, e.g., Watson et al, “The Molecular Biology of the Gene,” 4^{th} ed., Vols. 1 & 2, Benjamin, Menlo Park, Calif. (1987).

As used to refer to nucleic acid sequences, the term “homologous” indicates that two or more nucleotide sequences share a majority of their sequence. Generally, this will be at least about 70% of their sequence and preferably at least 95% of their sequence. Another indication that sequences are substantially identical is if they hybridize to the same nucleotide sequence under stringent conditions (see, e.g., Sambrook et al., “Molecular Cloning—A Laboratory Manual,” Cold Spring Harbor Laboratory, Cold Spring Harbor, N.Y. (1985)). Stringent conditions are sequence-dependent and will be different in different circumstances.

As used to refer to proteins, polypeptides, or peptides, which terms are often used interchangeably here, the term “homologous” is meant to indicate two proteins or polypeptides share a majority of their amino acid sequences. Generally, this will be at least 90% and usually more than about 95%. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

As used herein the term “isolated” is meant to describe a compound of interest (e.g., a polynucleotide or a polypeptide) that is in an environment different from that in which the compound naturally occurs. “Isolated” is meant to include compounds that are within samples that are substantially enriched for the compound of interest and/or in which the compound of interest is partially or substantially purified.

The term “isolated” as applied to, for example, nucleic acids, means a nucleic acid substantially separated from other macromolecules, cellular components, or DNA sequences which naturally accompany a native nucleic acid, e.g., ribosomes, polymerases, other nucleic acid sequences, and the like. The term includes a nucleic acid or polypeptide that has been removed from its naturally occurring environment, and includes recombinant or cloned DNA isolates and chemically synthesized analogues, and analogues biologically synthesized by heterologous systems. A substantially pure or biologically pure nucleic acid includes isolated forms of the nucleic acid.

The phrase “biologically pure” or “substantially pure” refers to material that is substantially or essentially free from components that normally accompany it as found in its native state, e.g., at least 60% free, preferably 75% free, and most preferably 90% free from other components with which it is naturally associated.

The term “recombinant” refers to a nucleic acid sequence that is not naturally occurring, or is made by the artificial combination of two otherwise separated segments of sequence, i.e., by chemical synthesis, genetic engineering, and the like.

The term “treatment” or “treating” means any therapeutic intervention in a mammal, preferably a human or bovine, including: (i) prevention, that is, causing the clinical symptoms not to develop, e.g., preventing infection from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by an infection.

Treatment is generally applied to any mammal susceptible to of having an S. aureus infection (e.g., mammals, birds, etc.), generally a mammal, usually a human or bovine where the treatment can be applied for prevention of bacterial infection of for amelioration of active bacterial infection, where the bacteria is a Staphylococcus bacteria, specifically S. aureus.

The terms “effective amount”, “therapeutic amount” and/or “therapeutic dosage” mean a dosage sufficient to provide treatment for the disease state being treated. This will vary depending on the patient, the disease and the treatment being effected. In the case of a bacterial infection, an “effective amount” is that amount necessary to substantially improve the likelihood of treating the infection, in particular that amount which improves the likelihood of successfully preventing infection or eliminating infection when it has occurred. In this context, “treatment” may include maintaining the status quo, or retarding progress of a disease state, sufficient to permit natural or artificial means to effect reduction or elimination of infection.

The term “protein” as used herein is intended to encompass any amino acid sequence and include modified sequences (e.g., glycosylated, PEGylated, containing conservative amino acid substitutions, fragments, etc.). The term includes naturally occurring (e.g., non-recombinant) proteins, polypeptides, peptides (particularly those isolated from a Staphylococcus bacteria, more particularly from S. aureus), and oligopeptides, as well as those which are recombinantly or synthetically synthesized according to methods well known in the art. As used in connection with the present invention, the term “protein” is specifically intended to cover naturally occurring proteins which occur in Staphylococcus spp. and useful in treating infection or in generating antibodies useful in treating infection. Where “polypeptide” or “protein” are recited herein to refer to an amino acid sequence of a naturally-occurring protein molecule, “polypeptide,” “protein,” and like terms are not meant to limit the amino acid sequence to the complete, native amino acid sequence associated with the recited protein molecule. In addition, the polypeptides and proteins of the invention, or fragments thereof, can be generated in synthetic form having D-amino acids rather than the naturally occurring L-amino acids.

“Polynucleotide” as used herein refers to an oligonucleotide, nucleotide, and fragments or portions thereof, as well as to peptide nucleic acids (PNA), fragments, portions or antisense molecules thereof, and to DNA or RNA of genomic or synthetic origin which can be single- or double-stranded, and represent the sense or antisense strand. Where “polynucleotide” is used to refer to a specific polynucleotide sequence (e.g., a RAP protein-encoding polynucleotide), “polynucleotide” is meant to encompass polynucleotides that encode a protein that is functionally equivalent to the recited protein, e.g., polynucleotides that are degenerate variants (i.e., variants in nucleic acid sequence that encode the same amino acid sequence and exist due to the degeneracy of the genetic code) or polynucleotides that encode biologically active variants or fragments of the recited protein.

“Antisense polynucleotide” means a polynucleotide having a nucleotide sequence complementary to a given polynucleotide sequence, including polynucleotide sequences associated with the transcription or translation of the given polynucleotide sequence (e.g, a promoter) and/or to a coding sequence of the given polynucleotide sequence, where the antisense polynucleotide is capable of hybridizing to a polynucleotide sequence. Of particular interest are antisense polynucleotides capable of inhibiting transcription and/or translation, either in vitro or in vivo.

“Peptide nucleic acid” as used herein refers to a molecule which comprises an oligomer to which an amino acid residue, such as lysine, and an amino group have been added. These small molecules, also designated anti-gene agents, stop transcript elongation by binding to their complementary (template) strand of nucleic acid (Nielsen et al., Anticancer Drug Des. 8:53-63 (1993)).

The term “antibody” is meant to refer to an immunoglobulin protein that is capable of binding an antigen. Antibody as used herein is meant to include the entire antibody as well as any antibody fragments (e.g., F(ab)′, Fab, Fv) or antibody CDR capable of binding the epitope, antigen, or antigenic fragment of interest. Preferred antibodies for assays and vaccines of the invention are immunoreactive or immunospecific for, and therefore specifically and selectively bind to, a protein of interest, e.g., an anti-RAP antibody. The term “antibody” encompasses all types of antibodies, e.g., polyclonal, monoclonal, humanized, chimeric, and those produced by the phage display methodology. Particularly preferred antibodies of the invention are antibodies that have a relatively high degree of affinity or avidity for RAP. An antibody of the invention is preferably immunoreactive with and immunospecific for a specific species, e.g., RAP obtained from S. aureus. An antibody exhibiting a high degree of avidity and affinity for RAP from S. aureus is a particularly preferred antibody of the invention.

“Antigenic fragment” of a protein is meant to refer to a portion of such a protein which is recognized by the immune system of the host (b-cells, t-cells and the like) such that it prompts the host to generate an immune response (cytokine expression and migration, antibody generation, etc.) that is effective not only against the fragment, but also against proteins and foreign substances that comprise the fragment or homologous structures.

“Binds specifically” means high avidity and/or high affinity binding of an antibody to a specific polypeptide, e.g., epitope of a protein, e.g., RAP protein. Antibody binding to its epitope on this specific polypeptide is preferably stronger than binding of the same antibody to any other epitope, particularly those that may be present in molecules in association with, or in the same sample, as the specific polypeptide of interest, e.g., binds more strongly to epitope fragments of a protein such as RAP so that by adjusting binding conditions the antibody binds almost exclusively to an epitope site or fragments of a desired protein.

“Detectably labeled antibody” means an antibody (or antibody fragment which retains binding specificity) having an attached detectable label. The detectable label is normally attached by chemical conjugation, but where the label is a polypeptide, it could alternatively be attached by genetic engineering techniques. The label may also be reflected in a ligand to which the antibody binds, such as a modified protein which bears the substrate for an enzyme which digests the substrate to give detectable moieties. The essence of a detectably labeled antibody is one whose presence is detectable by some means (electronic, calorimetric, chemiluminescent, fluorescent, etc.) to positively indicate the presence of a sought for target. Methods for production of detectably labeled proteins are well known in the art. Detectable labels known in the art include radioisotopes, fluorophores, paramagnetic labels, enzymes (e.g., horseradish peroxidase), or other moieties or compounds that either emit a detectable signal (e.g., radioactivity, fluorescence, color), or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies are well known in the art (e.g., “Antibodies: A Laboratory Manual,” Harlow et al., eds., Cold Spring Harbor Laboratory Press, Cold Spring Harbor, N.Y. (1988)).

The instant invention provides polypeptides for the prevention and treatment of S. aureus infections. The polypeptides of the invention are typically administered to hosts having or at risk of having a staphylococcal infection such as an S. aureus infection. The hosts are typically human patients. Animals also may be treated with the compositions of the invention, including but not limited to animals of commercial or veterinary importance, such as cows, sheep, and pigs, and experimental animals, such as rats, mice, or guinea pigs.

The compositions of the invention may be used prophylactically to prevent staphylococcal infections or may be therapeutically used after the onset of infection or the onset of symptoms associated with virulence. In some embodiments, induction of the formation of antibodies to the administered compound is desirable. In such instances, standard immunization protocols used in the art are preferred. The compositions administered for immunization may optionally include adjuvants.

In some embodiments of the invention, antagonists of RAP are provided. Such antagonists include but are not limited to antibodies that specifically bind to RAP; antibodies that specifically bind to a RAP ligand; ligands for RAP; and peptide, non-peptide, and peptidomimetic analogs of RAP and its ligands.

For therapeutic applications, “human” monoclonal antibodies having human constant and variable regions are often preferred so as to minimize the immune response of a patient against the antibody. Such antibodies can be generated by immunizing transgenic animals which contain human immunoglobulin genes (see Jakobovits et al., Ann. N.Y. Acad. Sci. 764: 525-35 (1995)). In connection with synthetic and semi-synthetic antibodies, such terms are intended to cover, but are not limited to, antibody fragments, isotype switched antibodies, humanized antibodies (e.g., mouse-human, human-mouse, and the like), hybrids, antibodies having plural specificities, fully synthetic antibody-like molecules, and the like.

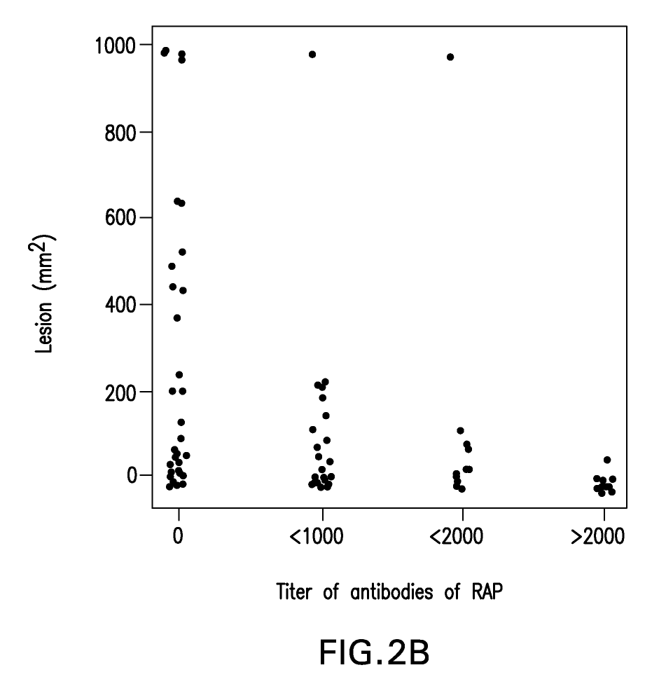

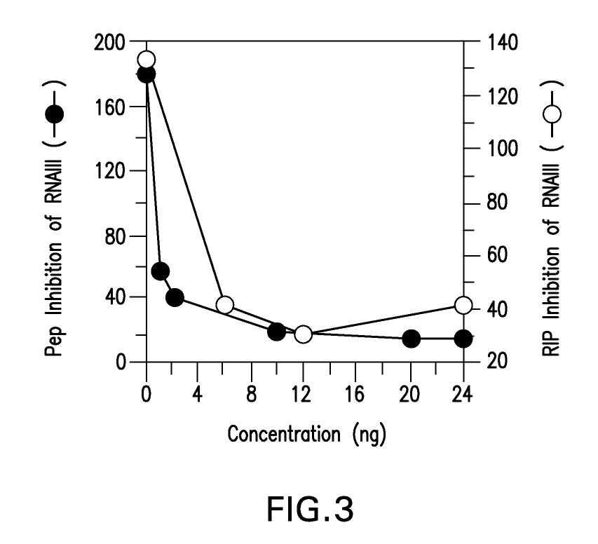

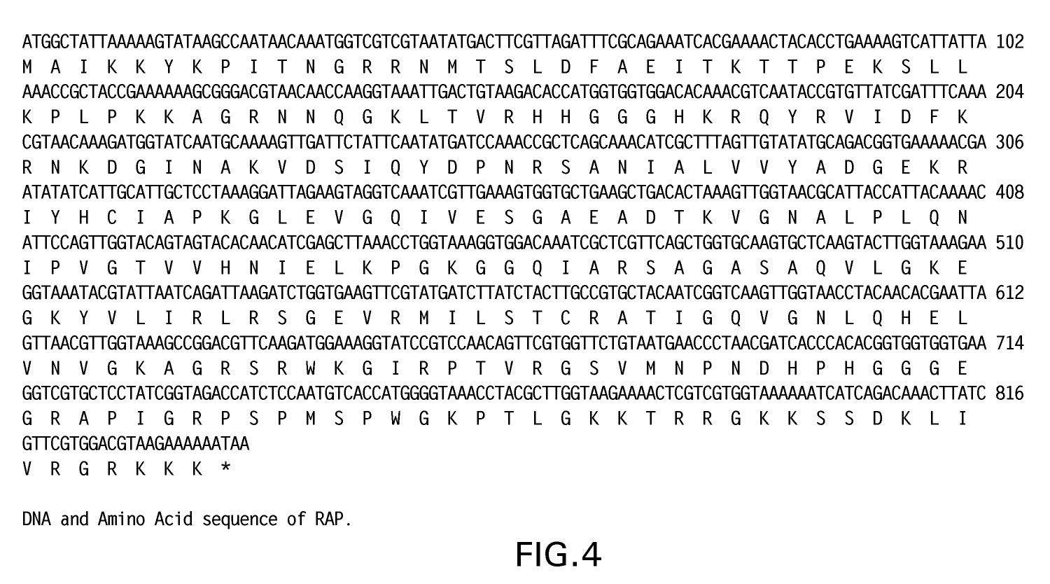

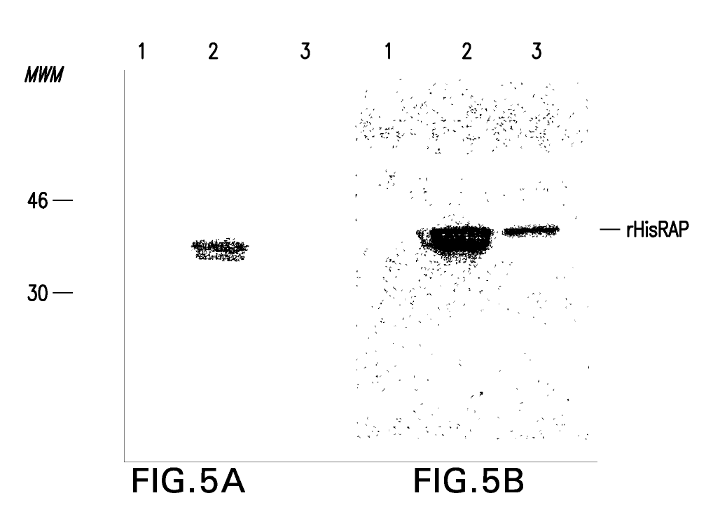

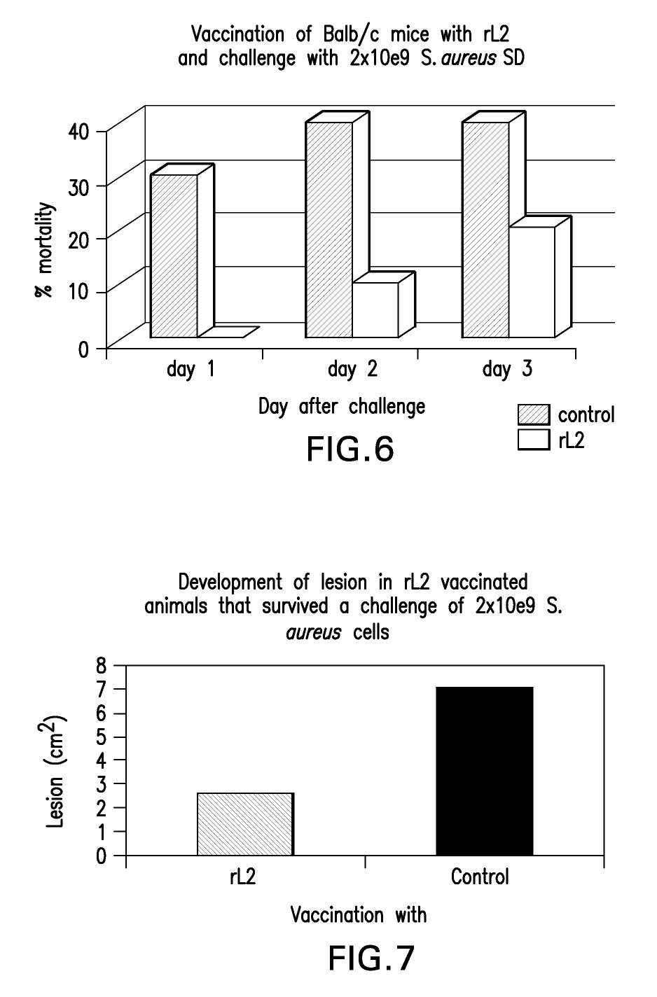

As discussed below, antibodies can be screened for the ability to block the binding of a ligand to RAP, RAP to its target molecule and/or for other properties, such as the ability to protect in vivo against S. aureus infection. RAP activates phosphorylation of TRAP or an intermediary thereof, which upregulates RNAIII expression. (Balaban et al., Activation and inhibition of the staphylococcal agr system. Response, Science, 287; 391a (2000); Korem et al., Transcriptional profiling of target of RNAIII-activating protein, a master regulator of staphylococcal virulence, Infect Immun. 73:6220-6228 (2005); Balaban et al., Regulation of Staphylococcus aureus pathogenesis via Target of RNAIII-Activating Protein (TRAP), J. Biol. Chem. 276, 2658-2667 (2001); Gov et al., Quorum sensing in staphylococci is regulated via phosphorylation of three conserved histidine residues, J Biol Chem. 279, 14665-14672 (2004). In applications co-pending herewith, the ability of RIP (See FIG. 3) to compete with RAP is addressed as a basis for therapeutic treatment and prevention of toxic infection by Staphylococcus aureus and other bacteria sharing this highly conserved pathway. The active agents of this invention prevent phosphorylation of TRAP by blocking the binding of RAP and consequent RNAIII production. Thus, antibodies and other ligands for RAP and TRAP are embraced as the active agents of this invention.

Candidate antagonists of RAP can be screened for function by a variety of techniques known in the art and/or disclosed within the instant application, such as protection against S. aureus infection in a mouse model. A multitude of appropriate formulations of the antagonists of the invention can be found in the formulary known to all pharmaceutical chemists: Remington's Pharmaceutical Sciences, 15^{th} ed., Mack Publishing Company, Easton, Pa. (1975), particularly Chapter 87, by Blaug et al., therein. These formulations include, for example, powders, pastes, ointments, jelly, waxes, oils, lipids, anhydrous absorption bases, oil-in-water or water-in-oil emulsions, emulsions, carbowax (polyethylene glycols of a variety of molecular weights), semi-solid gels, and semi-solid mixtures containing carbowax.

The quantities of active ingredient necessary for effective therapy will depend on many different factors, including means of administration, target site, physiological state of the patient, and other medicaments administered. Thus, treatment dosages should be titrated to optimize safety and efficacy. Typically, dosages used in vitro may provide useful guidance in the amounts useful for in situ administration of the active ingredients. Animal testing of effective doses for treatment of particular disorders will provide further predictive indication of human dosage. Various considerations are described, for example, in Goodman and Gilman's “The Pharmacological Basis of Therapeutics,” 7^{th} ed., MacMillan Publishing Company, New York (1985), and Remington's “Pharmaceutical Sciences,” 18^{th} ed., Mack Publishing Co, Easton Pa. (1990). Methods for administration are discussed therein, including oral, intravenous, intraperitoneal, intramuscular, transdermal, nasal, iontophoretic administration, and the like.

The compositions of the invention may be administered in a variety of unit dosage forms, depending on the method of administration. For example, unit dosage forms suitable for oral administration include solid dosage forms such as powder, tablets, pills, and capsules, and liquid dosage forms, such as elixirs, syrups, and suspensions. The active ingredients may also be administered parenterally in sterile liquid dosage forms. Gelatin capsules may contain the active ingredient and inactive ingredients, including powdered carriers, such as glucose, lactose, sucrose, mannitol, starch, cellulose or cellulose derivatives, magnesium stearate, stearic acid, sodium saccharin, talcum, magnesium carbonate and the like.

Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The concentration of the compositions of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The compositions of the invention may also be administered via liposomes. Liposomes include emulsions, foams, micelles, insoluble monolayers, liquid crystals, phospholipid dispersions, lamellar layers, and the like. A variety of methods are available for preparing liposomes, as described in, e.g., Szoka et al., Ann. Rev. Biophys. Bioeng. 9: 467 (1980), U.S. Pat. Nos. 4,235,871; 4,501,728; 4,837,028; and 5,019,369, incorporated herein by reference. In these preparations the composition of the invention to be delivered is incorporated as part of a liposome, alone or in conjunction with a molecule which binds to a desired target, such as antibody, or with other therapeutic or immunogenic compositions. Thus, liposomes either filled or decorated with a desired composition of the invention of the invention can be delivered systemically, or can be directed to a tissue of interest where the liposomes then deliver the selected therapeutic/immunogenic polypeptide compositions.

A liposome suspension containing a composition of the invention may be administered intravenously, locally, topically, etc. in a dose which varies according to, inter alia the manner of administration, the composition of the invention being delivered, and the stage of the disease being treated.

For solid compositions, conventional nontoxic solid carriers may be used, which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more compositions of the invention of the invention, and more preferably at a concentration of 25%-75%.

For aerosol administration, the compositions of the invention are preferably supplied in finely divided form along with a surfactant and propellant. Typical percentages of compositions of the invention are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides, may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

The constructs of the invention can additionally be delivered in a depot-type system, an encapsulated form, or an implant by techniques well-known in the art. Similarly, the constructs can be delivered via a pump to a tissue of interest.

Any of the foregoing formulations may be appropriate in treatments and therapies in accordance with the present invention, provided that the active agent in the formulation is not inactivated by the formulation and the formulation is physiologically compatible. Polyclonal and/or monoclonal antibodies to RAP and or TRAP are typically prepared as active agents of the present invention. The antigenic polypeptides of the invention for stimulating the expression of effective antibodies (active, as opposed to passive, protection) thereof may be prepared as described herein, and coupled to a carrier molecule, for example keyhole limpet hemocyanin, and injected into rabbits at selected times over several months. The rabbit sera may be tested for immunoreactivity to the polypeptides thereof. Monoclonal antibodies may be made by injecting mice with the polypeptides. Monoclonal antibodies may be screened by methods known in the art, as are described, for example, in Harlow et al., “Antibodies: A laboratory manual,” Cold Spring Harbor Press, New York (1988), and Goding “Monoclonal antibodies: Principles and Practice,” 2^{nd} ed., Academic Press, New York (1986). The antibodies will be tested for specific immunoreactivity with an epitope of the polypeptides. These antibodies will find use in diagnostic assays or as an active ingredient in a pharmaceutical composition.

For production of polyclonal antibodies, an appropriate target immune system is selected, typically a mouse or rabbit, although other species such as goats, sheep, cows, guinea pigs, and rats maybe used. The substantially purified antigen is presented to the immune system according to methods known in the art The immunological response is typically assayed by an immunoassay. Suitable examples include ELISA, RIA, fluorescent assay, or the like. These antibodies will find use in diagnostic assays or as an active ingredient in a pharmaceutical composition.

### RAP Protein

RAP protein can be produced by any suitable means, e.g., by isolation from a bacteria that naturally expresses RAP, by recombinant means (e.g., by expression of a polynucleotide having a sequence of SEQ ID NO:12), by synthetic means, and the like (Korem et al. (2005), supra.

In one embodiment, RAP is isolated directly from a strain of Staphylococcus producing RAP, e.g., S. aureus. Typically, wild-type cells are collected from post-exponential culture broth. Cells are then centrifuged and the supernatant subjected to purification by, for example, filtration followed by lyophilization, resuspension in water, and further purification (Balaban et al. (1998), supra; Yang et al. (2006), supra The staphylococci bacterium from which RAP may be isolated may include, but is not necessarily limited to, S. aureus, S. capitus, S. wameri, S. capitis, S. caprae, S. carnosus, S. saprophyticus, S. chronii, S. simulans, S. caseolyticus, S. epidermidis, S. haemolyticus, S. hominis, S. hyicus, S. kloosii, S. lentus, S. lugdunensis, S. scruri, and S. xylosus. Preferably RAP is isolated from S. aureus.

In another embodiment, RAP-encoding nucleic acid is employed to synthesize full-length RAP protein or fragments thereof, particularly fragments corresponding to functional domains (e.g., phosphorylation sites that interact with RAP, etc.); and including fusions of the subject polypeptides to other proteins or parts thereof. For expression, an expression cassette may be employed, providing for a transcriptional and translational initiation region, which may be inducible or constitutive, where the coding region is operably linked under the transcriptional control of the transcriptional initiation region, and a transcriptional and translational termination region. Various transcriptional initiation regions may be employed that are functional in the expression host.

The polypeptides may be expressed in prokaryotes or eukaryotes in accordance with conventional ways, depending upon the purpose for expression. For large scale production of the protein, a unicellular organism, such as E. coli, B. subtilis, S. cerevisiae, or cells of a higher organism such as vertebrates, particularly mammals, e.g., COS 7 cells, may be used as the expression host cells. Alternatively, RAP fragments can be synthesized.

With the availability of the polypeptides in large amounts, by employing an expression host, RAP protein can be isolated and purified in accordance with conventional ways, e.g., using HPLC, exclusion chromatography, gel electrophoresis, affinity chromatography, phage display or other purification techniques. The purified protein will generally be at least about 80% pure, preferably at least about 90% pure, and may be up to and including 100% pure.

The RAP proteins can be used for the production of vaccines or antibodies. For example, administration of the RAP protein in a recombinant, conjugated or in other known forms of vaccine administration will initiate an adaptive immune response resulting in the host's production of its own antibodies to RAP. Alternatively, short fragments provide for antibodies specific for the particular polypeptide, and larger fragments or the entire protein allow for the production of antibodies over the surface of the polypeptide, thereby using the RAP proteins to create antibodies for therapeutics. In particular, antibodies (monoclonal, polyclonal or humanized) may be raised (through typical and well known industry methods) to the wild-type or variant forms of RAP. Antibodies may be raised to isolated peptides corresponding to these domains, or to the native protein, e.g., by immunization with cells expressing RAP, immunization with liposomes containing RAP protein, etc. Moreover, the amino terminal of RAP has been shown to be similar to the structure of RIP. Considering the potent effect of RIP with respect to inhibition of the bacterium virulence cascade, the amino terminal of RAP likely plays a critical role in the functionality of the RAP protein. Therefore, antibodies raised and directed (through commonly known techniques in the art) specifically to the N-terminal of RAP will have an inhibitory effect on RAP function.

### Anti-RAP Antibodies and RAP Binding Proteins and Peptides

The present invention also provides an antibody that specifically binds and is immunoreactive (neutralizing to at least some degree) with RAP as well as disclosing peptides (Yang et al. (2003), supra) that may bind directly to RAP thereby inhibiting RAP's activity. With respect to the antibodies, they may be monoclonal, polyclonal or humanized, and will be prepared using methods well known in the art. In general, antibodies are prepared in accordance with conventional ways, where the protein or an antigenic portion thereof is used as an immunogen, by itself or conjugated to known immunogenic carriers, e.g., KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. In a preferred embodiment, the spleen or lymph node cells and myeloma cells are mixed in about 20:1 to about 1:1 ratio, but preferably in about 2:1 ratio. It is preferred that the same species of animal serve as the source of somatic and myeloma cells used in the fusion procedure, where the animal is chosen from rat, mouse, rabbit, cow, chicken, turkey, or man. The fusion of the somatic and myeloma cells produces a hybridoma, which is grown in culture to produce the desired monoclonal antibody by standard procedures. For further description, see, for example, “Monoclonal Antibodies: A Laboratory Manual,” Harlow et al., eds., Cold Spring Harbor Laboratories, Cold Spring Harbor, N.Y. (1988). If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage “display” libraries, usually in conjunction with in vitro affinity maturation.

The polyclonal antibodies of the present invention may be produced in a number of ways such as by injecting a rat, a mouse, a rabbit, a guinea pig, a goat, a cow, a chicken, or a turkey with RAP to initiate an immunogenic response. RAP may be coupled to a protein carrier such as keyhole limpet hemocyanin (KLH) or bovine serum albumin (BSA). An adjuvant may also be used. After a suitable amount of time to establish a high-titer of anti-RAP antibodies, the serum or eggs are collected. The presence of antibody in the serum or eggs may be tested by radioimmunoassay (RIA), by enzyme-linked immunosorbent assay (ELISA), or by immunoprecipitation. The immunoglobulins may be isolated by the sequential precipitation methods, by conventional methods of “salting out” the protein fractions from a salt solution, or by chromatographic methods well known to those skilled in the art.

### Identifying Agents Suitable for Treating Staphylococcus Infection

Of particular interest in the present invention is the identification of agents that have activity in affecting the expression and/or function of RAP. In general agents of interest are those that inhibit RAP activity, e.g., by inhibiting the ability of RAP to effect activation of RNAIII. Such agents are candidates for development of treatments for infection of pathogenic Staphylococcus. Of particular interest are screening assays for agents that have a low toxicity for human cells and/or high specificity for Staphylococcus, preferably with substantially no or little pressure for selection of strains resistant to the action of the agent, and without substantially affecting normal flora of the host (e.g., as distinguished from wide-spectrum antibiotics).

The term “agent” as used herein describes any binding molecule, e.g., protein or pharmaceutical, with the capability of altering RAP activity, by binding with some degree of affinity or avidity to RAP, or its target protein, TRAP. This invention focuses most strongly on antibodies, and antibody fragments, as agents. Non-antibody molecules may also be effective agents and can be identified by the protocols set forth herein. Generally a plurality of assay mixtures are run in parallel with different agent concentrations to detect differential responses to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, pheromones, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial (e.g., non-pathogenic Staphylococcus), fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Screening of Candidate Agents

A wide variety of in vitro assays may be used to screen candidate agents, including labeled in vitro binding assays, e.g., protein-protein binding, protein-DNA binding assays, electrophoretic mobility shift assays, immunoassays for protein binding, and the like. Purified naturally-occurring or recombinant RAP and RIP proteins, and/or synthetically produced peptides or fragments of RAP and/or RIP, can be used in various screening assays to identify ligands or substrates that bind to, modulate (e.g., increase or inhibit), or mimic the action of the native proteins. The purified proteins may also be used for determination of three-dimensional crystal structure, which can be used for modeling intermolecular interactions, transcriptional regulation, etc.

The screening assay can be a binding assay, wherein one or more of the molecules may be joined to a label, and the label directly or indirectly provides a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures. In general, the particular type of screening assay employed will preferably be one amenable to parallel, simultaneous screening of a large number of candidate agents.

Screening assays of the present invention encompass assays that examine the effect of candidate agents on the roles of RAP in RNAIII production and/or virulence factor production. For example, the candidate agent may be contacted with pathogenic Staphylococcus and the levels of RNAIII transcription in the presence of the agent compared to RNAIII transcription levels in the presence of RAP. Such screening assays can utilize recombinant host cells containing reporter gene systems, such as CAT (chloramphenicol acetyltransferase), β-galactosidase and the like, operably associated with RNAIII or virulence factor genes to facilitate detection of RNAIII or virulence gene transcription or to facilitate detection of RNAIII or virulence factor production. Alternatively, the screening assay can detect RNAIII or virulence factor transcription using hybridization techniques (e.g., Northern blot, PCR, etc.) well known in the art.

A variety of other reagents may be included in the screening assays described herein.

Where the assay is a binding assay, these include reagents like salts, neutral proteins, e.g., albumin, detergents, etc. that are used to facilitate optimal protein-protein binding, protein-DNA binding, and/or reduce non-specific or background interactions. Reagents that improve the efficiency of the assay, such as protease inhibitors, nuclease inhibitors, antimicrobial agents, etc. may be used. The mixture of components is added in any order that provides for the requisite binding. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

### Screening of Candidate Agents in an Animal Model

Agents having a desired activity as determined in the assays described above can be further screened for their ability to affect Staphylococcus virulence factor production, and to affect Staphylococcus infection, in a non-human animal model. The animal model selected will vary with a number of factors including, but not limited to, the particular pathogenic strain of Staphylococcus against which candidate agents are to be screened, the ultimate host for which the candidate agents are to serve as therapeutics, etc. Animals suitable for use in screening assays include any animal susceptible to infection by the selected Staphylococcus species. For example, where the Staphylococcus species is S. aureus, the animal model can be a rodent model, preferably a mouse model.

In general, the candidate agent is administered to a non-human animal susceptible to Staphylococcus infection, where the animal has been previously infected with Staphylococcus or receives an infectious dose of Staphylococcus in conjunction with the candidate agent. Preferably, the animal has no detectable RAP antibodies. The candidate agent can be administered in any manner desired and/or appropriate for delivery of the agent in order to affect a desired result. For example, the candidate agent can be administered by injection (e.g., by injection intravenously, intramuscularly, subcutaneously, or directly into the tissue in which the desired affect is to be achieved), topically, orally, or by any other desirable means. Normally, this screen will involve a number of animals receiving varying amounts and concentrations of the candidate agent (from no agent to an amount of agent that approaches an upper limit of the amount that can be delivered successfully to the animal), and may include delivery of the agent in different formulations. The agents can be administered singly or can be combined in combinations of two or more, especially where administration of a combination of agents may result in a synergistic effect.

The effect of agent administration upon the animal model can be monitored by any suitable method, such as assessing the number and size of Staphylococcus-associated lesions, overall health, etc. Where the candidate agent affects Staphylococcus infection in a desirable manner (e.g., by reducing infectious load, facilitating lesion regression, etc.), the candidate agent is identified as an agent suitable for use in treatment of Staphylococcus infection.

### Analysis of Cows for Anti-RAP Antibodies

Serum was collected from lactating dairy cows with one or more positive milk cultures for S. aureus (positive) and from lactating cows that have no record of having clinical case of S. aureus mastitis through one lactation (negative) and from calves which are 1 month and 4 months old. Sera were tested for anti-RAP antibodies by western blotting against post exponential supernatants of wild type S. aureus-containing RAP. Only 10% ( 2/20) of S. aureus-positive cows contain anti-RAP antibodies, while 63% ( 7/11) of S. aureus-negative cows contain anti-RAP antibodies. 38-46% of the calves contained anti-RAP antibodies. S. aureus-negative cows as well as calves will be followed in the future for correlation between titer of anti-RAP antibodies and natural infection rates. 60% ( 12/20) of the positive cows as compared to 18% ( 2/11) of the negative cows also contained antibodies to various unidentified proteins in S. aureus supernatant (presumed to be antibodies to other S. aureus proteins).

Thus, these data indicate that a majority of dairy cows that are negative for S. aureus mastitis naturally contain anti-RAP antibodies. These results support the use of RAP as a useful vaccine target site for the prevention of staphylococcal infections.

TABLE 1

| | | | |
|---|---|---|---|
| n | RAP | other S. aureus proteins | no anti-S. aureus |

Anti-RAP Antibodies in Cows

| | | | | |
|---|---|---|---|---|
| Cow | | | | |
| negative | 11 | 7 (63%) | 2 (18%) | 1 (18%) |
| positive | 20 | 2 (10%) | 12 (60%) | 6 (30%) |

Anti-RAP Antibodies in Calves

| | | | | |
|---|---|---|---|---|
| Calve | | | | |
| 1 month old | 20 | 5 (38%) | 2 (10%) | 13 (65%) |
| 4 months old | 20 | 6 (46) | 1 (5) | 13 (65%) |

### Identified Candidate Agents

The compounds having the desired pharmacological activity may be administered in a physiologically acceptable carrier to a host for treatment of pathogenic Staphylococcus infection. The therapeutic agents may be administered in a variety of ways, orally, topically, parenterally, e.g., subcutaneously, intraperitoneally, intravascularly, intrapulmonary (inhalation), etc. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### Treating Staphylococcus Infection

The invention provides a method for preventing or treating a human or an animal susceptible to infection by a pathogenic Staphylococcus (e.g., S. aureus in humans) by administering an agent that inhibits RAP activity in facilitating virulence factor production, e.g., by inhibition RAP-mediated activation of RNAIII and subsequent virulence factor production.

In one embodiment, the host is treated by administration of RAP inhibitor, such as an anti-RAP antibody or a RAP binding peptide, or all of the foregoing. In one embodiment the RAP inhibitor is co-administered with other RAP inhibitors and/or co-administered with other inhibitors of S. aureus virulence factor production. Combination therapies (e.g., administration of multiple RAP inhibitory agents) may involve co-administration or sequential administration of the active components. The dosage regimen may be adjusted to provide the optimum therapeutic response. For example, several divided doses may be administered daily or the dose may be proportionally reduced as indicated by the therapeutic situation. The active compounds may be administered in any convenient manner, such as by oral, intravenous, intramuscular, subcutaneous, buccal, transdermal, or inhalation routes.

Formulations composed of RAP inhibitor are administered at a therapeutically effective dosage, e.g., a dosage sufficient to improve the chance of successful prevention or treatment of infection. Administration of such a formulation can be via any of the accepted modes of administration for agents that serve similar utilities, preferably by systemic administration.

Human dosage levels for treating infections are known and generally include a daily dose from about 0.1 to 500.0 mg/kg of body weight per day, preferably about 6.0 to 200.0 mg/kg, and most preferably about 12.0 to 100.0 mg/kg. Generally, it is sought to obtain a serum concentration of such a formulation approximating or greater than circulating levels needed to reduce or eliminate any infection in less than 10 days. For administration to a 70 kg person, the dosage range would be about 50 mg to 3.5 g per day, preferably about 100 mg to 2 g per day, and most preferably about 200 mg to 1 g per day. The amount of formulation administered will, of course, be dependent on the subject and the severity of the affliction, the manner and schedule of administration, and the judgment of the prescribing physician.

In employing formulation for treatment of infections, any pharmaceutically acceptable mode of administration can be used. The formulations can be administered either alone or in combination with other pharmaceutically acceptable excipients, including solid, semi-solid, liquid or aerosol dosage forms, such as, for example, tablets, capsules, powders, liquids, gels, suspensions, suppositories, aerosols or the like. The formulations can also be administered in sustained or controlled release dosage forms (e.g., employing a slow release bioerodable delivery system), including depot injections, osmotic pumps, pills, transdermal and transcutaneous (including electrotransport) patches, and the like, for prolonged administration of a predetermined rate, preferably in unit dosage forms suitable for single administration of precise dosages.

The compositions will typically include a conventional pharmaceutical carrier or excipient and a formulation of the invention. In addition, these compositions may include other active agents, carriers, adjuvants, etc. Generally, depending on the intended mode of administration, the pharmaceutically acceptable composition will contain about 0.1% to 90%, preferably about 0.5% to 50%, by weight of active compound, the remainder being suitable pharmaceutical excipients, carriers, etc. Actual methods of preparing such dosage forms are known, or will be apparent, to those skilled in this art. For example, see Remington's “Pharmaceutical Sciences,” Mack Publishing Company, Easton, Pa., 15^{th} ed. (1975).

Parenteral administration is generally characterized by injection, either subcutaneously, intradermally, intramuscularly, or intravenously, preferably subcutaneously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain certain amounts of non-toxic auxiliary substances, such as wetting or emulsifying agents, pH buffering agents, solubility enhancers, and the like, such as for example, sodium acetate, sorbitan monolaurate, triethanolamine oleate, cyclodextrins, and the like.

The percentage of active ingredient contained in such parenteral compositions is highly dependent on the specific nature thereof, as well as the needs of the subject. However, percentages of active ingredient of 0.01% to 10% in solution are employable, and will be higher if the composition is a solid which will be subsequently diluted to the above percentages. Preferably, the composition will comprise 0.2-2% of the active ingredient in solution.

A more recently devised approach for parenteral administration employs the implantation of a slow-release or sustained-release system, such that a constant level of dosage is maintained. Various matrices (e.g., polymers, hydrophilic gels, and the like) for controlling the sustained release, and for progressively diminishing the rate of release of active agents are known in the art. See U.S. Pat. No. 3,845,770 (describing elementary osmotic pumps); U.S. Pat. Nos. 3,995,651; 4,034,756; and 4,111,202 (describing miniature osmotic pumps); U.S. Pat. Nos. 4,320,759 and 4,449,983 (describing multichamber osmotic systems referred to as push-pull and push-melt osmotic pumps); and U.S. Pat. No. 5,023,088 (describing osmotic pumps patterned for the sequentially timed dispensing of various dosage units).

Formulations of active components may also be administered to the respiratory tract as a nasal or pulmonary inhalation aerosol or solution for a nebulizer, or as a microfine powder for inhalation, alone or in combination with an inert carrier, such as lactose, or with other pharmaceutically acceptable excipients. In such a case, the particles of the formulation may advantageously have diameters of less than 50 microns, preferably less than 10 microns. See, e.g., U.S. Pat. No. 5,364,838, which discloses a method of administration for insulin that can be adapted for the administration of formulations of the present invention.

### Vaccination

The invention provides a vaccine for inoculating a human or an animal susceptible to infection by a pathogenic Staphylococcus (e.g., S. aureus) by administering RAP, or an antigenically effective portion of RAP, in a pharmaceutically acceptable carrier, which may optionally comprise an adjuvant. Formulations appropriate for elicitation of the immune response are well known in the art. In general, the host is exposed to the antigen, such as RAP, which perturbs the host's immune system and results in an immune response towards the antigen. An adjuvant can be added with the antigen to increase the immune response to the antigen. The amount of polypeptide administered is an amount sufficient to elicit a protective immune response in the host. Methods for determining such appropriate amounts are routine and well known in the art. For example, RAP and/or antigenically effective portion(s) thereof can be used to vaccinate an animal model of Staphylococcus infection. The amounts effective in such animal models can be extrapolated to other hosts (e.g., livestock, humans, etc.) in order to provide for an amount effective for vaccination. The same method can be used to address existing nascent infections in the host-vaccination and treatment may occur simultaneously.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of eliciting a protective immune response to Staphylococcus, where the Staphylococcus has the ability to cause an infection in a mammalian host and release virulence factors in the host, comprising administering to the host an RNAIII activating protein (RAP) polypeptide in an amount effective to elicit a an immune response in the host sufficient to reduce the rate of RAP's upregulation of the virulence factors in the host.

2. The method of claim 1 wherein the RAP protein is comprised of the entire RAP amino acid sequence.

3. The method of claim 1 wherein the RAP protein is comprised of an antigenic fragment that includes less than the entire amino acid sequence of RAP wherein said RAP polypeptide has an amino terminal.

4. The method of claim 3 wherein the RAP protein is comprised of an antigenic fragment that includes at least twenty amino acids including the amino terminal portion of the RAP protein.

5. A pharmaceutical composition comprising an anti-RAP antibody in a suitable pharmaceutical carrier to treat a condition or a disease in a mammalian host caused by Staphylococcus, wherein the anti-RAP antibody binds to and neutralizes biological activity of RAP and the anti-RAP antibody is present in an amount suitable for administration to the host in a dosage range that is therapeutically effective for treating the condition or disease.

6. The pharmaceutical composition of claim 5 wherein the antibody is monoclonal or polyclonal.

7. The pharmaceutical composition of claim 5 wherein the antibody is humanized.

8. An isolated antibody or antigen binding fragment which antibody or fragment binds RAP circulating in a mammalian host with sufficient neutralizing effect to inhibit release of virulence factors by Staphylococcus infecting said host.

9. The isolated antibody or antigen binding fragment of claim 8 wherein the isolated antibody or fragment binds to the RAP polypeptide at its amino terminal wherein said RAP polypeptide is comprised of an amino acid sequence having an amino terminal and a carboxyl terminal, and wherein said antibody binds to an epitope found within twenty amino acids of said amino terminal.

10. The antibody of claim 8, which is a monoclonal antibody.

11. The antibody of claim 8, which is a humanized antibody.

12. The antibody of claim 8, which is an antibody fragment.

13. The antibody fragment of claim 12, which comprises a Fab fragment.

14. The antibody of claim 8, which is an agonist antibody.

15. The antibody of claim 14, which is a humanized antibody.

16. A composition comprising the antibody of claim 8 and an acceptable carrier.

17. The antibody of claim 8, which is an antagonist antibody.

18. The antibody of claim 17, which is a humanized antibody.

19. The antibody of claim 17, which is an antibody fragment.

20. A method of providing passive immunity to a mammalian host to Staphylococcus, where the Staphylococcus has the ability to cause an infection in said host and release virulence factors in the host, comprising administering an agent to the host that inhibits the release of Staphyloccocal virulence factors in the host by binding to RNAIII activating protein (RAP), which inhibits RAP from upregulating Staphylococcus virulence factors and where the agent is an antibody or a binding fragment.
